(19) United States
(12) Patent Application Publication
(10) Pub. No.: US 2003/0113934 A1
(43) Pub. Date: Jun. 19, 2003

(54) Diagnostic sensing apparatus

(76) Inventor: Sung-Yun Kwon, Fremont, CA (US)

Correspondence Address: Thomas P. McCracken, POWDERJECT TCHNOLOGIES INC., 6511 Dumbarton Circle, Fremont, CA, 94555, US

(21) Appl. No.: 10/023,104

(22) Filed: Dec. 17, 2001

(51) Int. Cl. G01N 21/00; A61B 5/00

(52) U.S. Cl. 436/164; 436/165; 436/169; 436/172; 435/14; 422/56; 422/57; 422/82.05; 422/82.08; 600/310; 600/316; 600/317; 600/365; 600/362; 600/573; 600/576; 600/583; 600/584

(57) ABSTRACT

A sensing apparatus and methods for measuring or detecting an analyte present in a biological system are provided. The methods entail use of the sensing apparatus that contains a reporter system specific for the analyte of interest, where the reporter system is either affixed to a planar backing or attached to particles that are delivered to the superficial layers of the skin. The reporter system includes a reporting reagent that absorbs or emits a detectable radiation and is placed in communication with the analyte, or in communication with tissue or body fluids suspected of containing the analyte. The sensing apparatus is illuminated, and a radiation signal from the reporting reagent is measured or detected and then associated with the presence or quantity of analyte.

### CROSS-REFERENCE TO RELATED APPLICATIONS

Not Applicable

### FIELD OF THE INVENTION

The invention relates generally to an apparatus and methods of monitoring the presence and/or concentration of a target analyte present in an aqueous biological system. More particularly, the invention relates to an apparatus and methods for determining the presence or measuring the concentration of one or more analytes in a transdermally accessed sample. One important application of the invention involves an apparatus and method for monitoring blood glucose using non-invasive or minimally invasive monitoring techniques.

### BACKGROUND OF THE INVENTION

This application relates to an apparatus and methods for detecting and quantifying analytes in body fluids using fluorescence techniques.

Various types of apparatus are currently used to measure analytes. They include pads, membrane dipsticks, swabs, tubes, vials, cuvettes, and capillaries. Reagents for determining the presence or concentration of specific analytes may be present in or added to these devices to measure the analyte of interest. For example, dipsticks containing reagents, that measure hormones, are useful in determining whether the user is pregnant.

Numerous methods for detecting and quantifying analytes in body fluids are known. These tests typically rely on physiological fluid samples removed from a subject, either using a syringe or by pricking the skin. For example, in the case of glucose these methods include various calorimetric reactions, measuring a spectrophotometric change in the property of any number of products in a glycolytic cascade or measuring the oxidation of glucose using a polarimetric glucose sensor.

Diabetes is a major health concern, and treatment of the more severe form of the condition, Type I (insulin-dependent) diabetes, requires one or more insulin injections per day. Insulin controls utilization of glucose or sugar in the blood and prevents hyperglycemia, which, if left uncorrected, can lead to ketosis. On the other hand, improper administration of insulin therapy can result in hypoglycemic episodes, which can cause coma and death. Hyperglycemia in diabetics has been correlated with several long-term effects, such as heart disease, atherosclerosis, blindness, stroke, hypertension and kidney failure.

The value of frequent monitoring of blood glucose as a means to avoid or at least minimize the complications of Type I diabetes is well established. According to the National Institutes of Health, glucose monitoring is recommended 4-6 times a day. Patients with Type II (non-insulin-dependent) diabetes can also benefit from blood glucose monitoring in the control of their condition by way of diet and exercise.

Conventional blood glucose monitoring methods generally require the drawing of a blood sample (e.g., by finger prick) for each test, and a determination of the glucose level using an instrument that reads glucose concentrations by electrochemical or calorimetric methods. Type I diabetics must obtain several finger prick blood glucose measurements each day in order to maintain tight glycemic control. However, the pain and inconvenience associated with this blood sampling has lead to poor patient compliance, despite strong evidence that tight control dramatically reduces long-term diabetic complications. In fact, these considerations can often lead to an abatement of the monitoring process by the diabetic.

To satisfy the need for simpler and less painful sensing and monitoring needs of the population, this invention provides for a simple sensing apparatus and methods of monitoring for the presence of analytes in body fluids. The methods are non-invasive or minimally invasive and have little or no pain associated with the monitoring steps helping to increase patient compliance.

### BRIEF SUMMARY OF THE INVENTION

The present invention provides apparatus and methods for sampling an analyte present in a biological system. Accordingly, it is a primary object of the present invention to provide a sensing apparatus. The apparatus comprises a substantially planar occlusive backing and a reporter system. The reporter system absorbs or emits a detectable radiation, and is attached, coupled, adhered, or otherwise connected to a first planar surface of the occlusive backing. The reporter system binds an analyte of interest, and the ability of the reporter system to absorb or emit radiation is detectably altered in a concentration-dependent manner when the analyte is bound to the reporter system. It is preferable that the reporter system is attached to an occlusive backing that has sufficient drape characteristics to allow for positioning of the apparatus over an uneven skin or mucosal surface, particularly allowing the apparatus to be positioned over a surface on a limb or other body part and remain in place despite normal bodily movements and/or physical changes (e.g., perspiration) affecting the surface.

In one aspect of the invention, the reporter system comprises a specific binding pair having a first component that is an analyte-specific binding ligand comprising a first light-absorbing material, and a second component that binds to the binding ligand of said first component and comprises a second light-absorbing material. Binding of the second component to the first component is reversible, and the analyte binds to the first component in a competitive manner, thereby displacing the second component. In turn, displacement of the second component produces a detectable alteration in the energy transfer between the first component and the second component, wherein such alteration is proportional to the concentration or amount of said analyte that binds to the first component. In certain embodiments, the binding ligand can be a glucose binding ligand, and the analyte of interest is glucose. More particularly, the glucose binding ligand can be concanavalin-A, and the second component of the reporter system can comprise a dextran glycoconjugate. The detectable alteration in the energy transfer between the first component and the second component can comprise a non-radiative fluorescence resonance energy transfer between the first and second light-absorbing materials, and in certain preferred embodiments, the first component of the specific binding pair is tetramethylrhodamine isothiocyanate-concanavalin A (“TRITC-ConA”) and the second component of the specific binding pair is fluorescein isothiocyanatedextran (“FITC-dextran”).

It is also a primary object of the present invention to provide a method for detecting the presence or amount of an analyte present beneath a target skin or mucosal surface of an individual. The method entails: (a) disrupting the target surface to create one or more passages in that surface sufficient to allow said analyte to flow, exude, diffuse or otherwise pass from beneath the target surface to the target surface; (b) placing a sensing apparatus constructed according to the present invention in contact with the target surface; and (c) detecting an alteration in the ability of the reporter system to absorb or emit radiation, thereby obtaining a signal indicative of the presence and/or amount of analyte present beneath the target surface.

In certain aspects of the invention, the target surface is disrupted by accelerating small particles into the target surface. Such particles typically have a size ranging from about 0.1 to 250 microns (nominal diameter). In certain preferred embodiments, the particles have a size ranging from about 10 to 70 microns. It is also preferred that the analyte of interest is glucose.

It is yet another primary object of the present invention to provide a method for quantifying glucose present in a body fluid beneath a target surface. The method entails: (a) accelerating particles into the target surface, wherein acceleration of such particles into the target surface is effective to allow passage of glucose from beneath the target surface to the target surface; (b) contacting the glucose present at the target surface with a specific binding pair comprising a first component which is a glucose binding ligand containing a first light-absorbing material, and a second component which is a glycoconjugate containing a second light-absorbing material. The excited state energy level of the first light-absorbing material overlaps with the excited state energy level of the second light-absorbing material, and the ligand and glycoconjugate pair is chosen such that they reversibly bind to each other thereby allowing glucose present at the target surface to displace the glycoconjugate and competitively bind to the ligand; (c) determining the extent to which non-radiative fluorescence resonance energy transfer occurs between the first light-absorbing and the second light-absorbing material in the presence of the glycoconjugate displaced by glucose and the ligand reversibly bound to glucose; and (d) comparing the result of step (c) with the relationship between the extent of non-radiative energy transfer between the first light-absorbing material and the second light-absorbing material and glucose concentration in the body fluid determined in a calibration step.

In the practice of the method, acceleration of the particles into the target surface serves to permeabilize the target surface. In certain aspects, the particles are accelerated toward the target surface using a particle injection device (needleless syringe).

It is also a primary object of the invention to provide a method for detecting the presence or amount of an analyte present beneath a target skin surface of an individual. The method entails: (a) providing a particulate reporter system, wherein the reporter system binds the analyte of interest and the ability of said reporter system to absorb or emit radiation is altered in a concentration-dependent manner when the analyte is bound to the reporter system, and the particulate reporter system is comprised of a homogenous population of particles each having a size ranging from 0.1-250 microns; (b) administering the reporter system into the target skin surface such that the particulate reporter system is delivered to a substantially uniform and homogenous depth within the skin; (c) allowing the reporter system to contact the analyte; and (d) detecting an alteration in the ability of the reporter system to absorb or emit radiation thereby obtaining a signal indicative of the presence or amount of analyte present beneath the target skin surface.

It is preferred that the particulate reporter system is delivered using a particle injection device (needleless syringe), and that the particles are delivered at a depth of about 1-50 microns below the target surface. Although a number of particle sizes will be suitable for use in the method, it is preferable that the particles are provided in a homogenous size, and that they have a size ranging from about 10 to 70 microns.

In one aspect, the method is practiced using a reporter system that comprises a specific binding pair having a first component that is an analyte-specific binding ligand and includes a first light-absorbing material, and a second component that binds to the binding ligand of the first component and includes a second light-absorbing material. The binding of the second component to the first component is reversible, and the analyte binds to the first component in a competitive manner, thereby displacing the second component. In turn, the displacement of the second component produces a detectable alteration in the displacement of the second component and produces a detectable alteration in the energy transfer between the first component and the second component, wherein such alteration is proportional to the concentration or amount of the analyte that binds to the first component. In certain embodiments, the binding ligand can be a glucose binding ligand, and the analyte of interest is glucose. More particularly, the glucose binding ligand can be concanavalin-A, and the second component of the reporter system can comprise a dextran glycoconjugate. The detectable alteration in the energy transfer between the first component and the second component can comprise a non-radiative fluorescence resonance energy transfer between the first and second light-absorbing materials, and in certain preferred embodiments, the first component of the specific binding pair is tetramethylrhodamine isothiocyanate-concanavalin A (“TRITC-ConA”) and the second component of the specific binding pair is fluorescein isothiocyanate-dextran (“FITC-dextran”). These components are fluorophore labeled ligands formed by reaction of the parent lectin, concanavalin-A and a glycoconjugated dextran with the respective dyes reactive by virtue of having isothiocyanate moieties.

In the above-described methods, the analyte can be any specific substance or component that one is desirous of detecting and/or measuring in a chemical, physical, enzymatic, or optical analysis. Such analytes include, but are not limited to, toxins, contaminants, amino acids, enzyme substrates or products indicating a disease state or condition, other markers of disease states or conditions, drugs of recreation and/or abuse, performance-enhancing agents, therapeutic and/or pharmacologic agents, electrolytes, physiological analytes of interest (e.g., calcium, potassium, sodium, chloride, bicarbonate (CO₂), glucose, urea (blood urea nitrogen), lactate, and hemoglobin), lipids, and the like. In preferred embodiments, the analyte is a physiological analyte of interest, for example glucose, or a chemical that has a physiological action, for example a drug or pharmacological agent. As will be understood by the ordinarily skilled artisan upon reading the present specification, there are a large number of analytes that can be sampled using the present invention.

An advantage of the invention is that the instant sampling processes can be readily practiced inside and outside of the clinical setting and without pain.

These and other objects, aspects, embodiments and advantages of the present invention will readily occur to those of ordinary skill in the art in view of the disclosure herein.

Before describing the present invention in detail, it is to be understood that this invention is not limited to particularly exemplified analytes or process parameters as such may, of course, vary. It is also to be understood that the terminology used herein is for the purpose of describing particular embodiments of the invention only, and is not intended to be limiting.

All publications, patents and patent applications cited herein, whether supra or infra, are hereby incorporated by reference in their entirety and for all purposes.

Definitions

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

It must be noted that, as used in this specification and the appended claims, the singular forms “a,” “an,” and “the” include plural referents unless the content clearly dictates otherwise. Thus, for example, reference to “a particle” includes a mixture of two or more such particles, reference to “an analyte” includes mixtures of two or more such analytes, and the like.

In describing the present invention, the following terms will be employed, and are intended to be defined as indicated below.

The term “analyte” is used herein in its broadest sense to denote any specific substance or component that is being detected and/or measured in a physical, chemical, biochemical, electrochemical, photochemical, spectrophotometric, polarimetric, calorimetric, or radiometric analysis. A detectable signal can be obtained, either directly or indirectly, from such a material. In preferred embodiments, the analyte is a physiological analyte of interest (e.g., a physiologically active material), for example glucose, or a chemical that has a physiological action, for example a drug or pharmacological agent. Examples include materials for blood chemistries (blood pH, pO₂, pCO₂, Na⁺, Ca⁺⁺, K⁺, lactic acid, glucose, and the like), for hematology (hormones, hormone releasing factors, coagulation factors, binding proteins, acylated, glycosylated, or otherwise modified proteins and the like), and immuno-diagnostics, toxins, contaminants, amino acids, enzymes, enzyme substrates or products indicating a disease state or condition, immunological substances, other markers of disease states or conditions, performance-enhancing agents, therapeutic and/or pharmacologic agents, electrolytes, physiological analytes of interest (e.g., calcium, potassium, sodium, chloride, bicarbonate ([HCO₂]⁻²), glucose, urea (blood urea nitrogen), lactate, and hemoglobin), materials for DNA testing, nucleic acids, proteins, carbohydrates, lipids, electrolytes, metabolites (including but not limited to ketone bodies such as 3-hydroxybutyric acid, acetone, and acetoacetic acid), therapeutic or prophylactic drugs, gases, compounds, elements, ions, drugs of recreation and/or abuse, anabolic, catabolic or reproductive hormones, anticonvulsant drugs, antipsychotic drugs, alcohol, cocaine, cannabinoids, opiates, stimulants, depressants, and their metabolites, degradation products and/or conjugates. The term “target analyte” refers to the analyte of interest in a specific monitoring method or technique.

The term “analogue” refers to a material that has at least some binding properties in common with those of the analyte such that there are ligands that bind to both. The analogue and the analyte, however, do not bind to each other. The analogue may be a derivative of the analyte such as a compound prepared by introducing functional chemical groups onto the analyte that do not affect at least some of the binding properties of the analyte. Another example of a derivative is a lower molecular weight version of the analyte that nonetheless retains at least some of the binding properties of the analyte.

As used herein, the term “pharmacological agent” intends any compound or composition of matter which, when administered to an organism (human or animal subject), induces a desired pharmacologic and/or physiologic effect by local and/or systemic action.

As used herein, the term “sampling” means access to and monitoring of a substance from any biological system from the outside, e.g., across a membrane such as skin or tissue. The membrane can be natural or artificial, and is generally animal in nature, such as natural or artificial skin, blood vessel tissue, intestinal tissue, and the like. A “biological system” thus includes both living and artificially maintained systems.

The term “individual” is used interchangeable herein with the term “subject,” and encompasses any warm-blooded animal, particularly including a member of the class Mammalia such as, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult, child and newborn subjects, whether male or female, as well as fetuses, are intended to be covered.

The term “sensing apparatus” encompasses any device that can be used to measure the concentration of an analyte of interest. A preferred sensing apparatus will be a substantially planar backing with a reporter system connected to it. The reporter system will measure the level of an analyte present in a body fluid. Preferred analytes will be found in interstitial fluid. Detection and/or quantification of a radiation signal can be carried out using readily available radiation emission/adsorption monitoring devices. Examples of fluorogenic systems include non-radiative energy transfer systems.

The term “non-radiative fluorescence resonance energy transfer” is used interchangeably with the acronym “FRET” herein. The process involves a transfer of energy from a first fluorescent reagent that acts as an energy donor to a second fluorescent reagent that acts as an energy acceptor.

The term “fluorescence reagent” is used interchangeably with the term “reporter system” and refers to a material whose fluorescence behavior (e.g., intensity, emission spectrum, or excitation spectrum) changes in the presence of the analyte being detected. In some embodiments, the fluorescent reagent binds reversibly to the analyte. For example, the reagent may be a fluorophore, or a compound labeled with a fluorophore, that binds directly to the analyte. It is the fluorescence behavior of this molecule (or compound labeled with this molecule) that changes as a result of analyte binding.

The reagent may also include more than one component. For example, it may include an analogue to the analyte labeled with a fluorophore and a ligand (e.g., an antibody, receptor for the analyte, lectin, enzyme, or lipoprotein) that binds competitively (and specifically) to the analogue and the analyte. In this case, it is the fluorescence behavior of the labeled analogue that changes as a result of ligand binding to analyte. Conversely, the ligand may be labeled, rather than the analogue, in which case it is the fluorescence behavior of the labeled ligand that changes.

The reagent may also include two components, one of which is labeled with an energy-absorbing donor molecule and the other of which is labeled with an energy-absorbing acceptor molecule; the donor and acceptor have overlapping excited state energy levels. One or both molecules forming the donor-acceptor pair can be fluorophores. Regardless, however, it is the fluorescence associated with the non-radiative resonance energy transfer from donor to acceptor that is measured. The components may be members of a specific binding pair (e.g., an analogue of the analyte and a ligand capable of binding competitively (and specifically) to both the analogue and the analyte) or ligands (e.g., antibodies or oligonucleotides) that bind specifically to different portions of the analyte.

FRET can also be measured where a single reagent capable of binding to the analyte is labeled with both donor and acceptor molecules.

The term “fluorophore” refers to a molecule that absorbs energy and emits light.

The tern “fluorescence” refers to radiation emitted in response to excitation by radiation of a particular wavelength. It includes short-lived (nanosecond range) and long-lived excited state lifetimes, the latter is sometimes referred to as phosphorescence.

### DETAILED DESCRIPTION OF THE INVENTION

The invention relates to a sensing apparatus and methods for sampling analytes present in a biological system, typically a physiologically active material that is present beneath a target skin or mucosal surface of an individual.

An ideal sensing apparatus should contain a reporting system and be capable of detecting a wide range of physiological concentrations of analyte. As used herein, “physiological concentration” refers to the concentration of analyte found in both normal and pathological states. For example, in the case of glucose it refers to glucose levels found in normal, hypoglycemic, and hyperglycemic patients. In the case of analytes not normally present in the biological system, the reporting system should be capable of detecting trace amounts of the substance.

The sensing apparatus should also be reliable, reusable and easy to use. In addition, the sensing apparatus should be non-invasive or minimally invasive.

The sensing apparatus can be constructed from a wide range of materials, including both rigid and pliable materials. Preferably, the apparatus is constructed of a planar material that is pliable such that it can mold to the surface to which it is applied. In one embodiment, the planar material is transparent so that light can be transmitted through the material from an external light source and light can be detected from beneath the material by an external detector. Ideally the light source and detector would be in a single unit.

The sensing apparatus is preferably made out of flexible material that is impervious to moisture. Such materials can include but not be limited to plastic or polymeric materials including thermoplastics such as polycarbonates, polyesthers (e.g., MYLAR™ and polyethylene terephthalate (PET)), polyvinyl chloride (PVC), polyethylene glycol hydrogel (PEGH), polyurethanes, polyethers, polyamides, polyimides, or copolymers of these thermoplastics, such as PETG (glycol-modified polyethylene terephthalate). Other suitable flexible, water impervious materials are well known to those of skill in the art. The material can be cut in a variety of shapes and sizes as required for the location that the sensing apparatus will be used and the volume of body fluid required for sampling the analyte of interest. The planar material will typically range from 0.01 up to 2 or more millimeters in thickness, depending upon the material used.

The sensing apparatus will have an adhesive component to at least one edge, or portion of an edge of the surface that comes in contact with the target skin or mucosal surface of an individual. Preferably the entire edge of the surface of the sensing apparatus that comes in contact with the target skin or mucosal surface will have an adhesive component for adhering the sensing apparatus to the individual. Alternatively, the entire surface of the sensing apparatus that comes in contact with the target skin or mucosal surface of the individual will be covered with the adhesive material. Typically the adhesive will be a pressure-sensitive adhesive. Pressure-sensitive adhesives generally comprise a pressure-sensitive adhesive component, a tackifier and softener. Examples of such pressure sensitive adhesive components include but are not limited to natural and synthetic resins such as natural rubber, polyisobutylene rubber, polybutadiene rubber, silicone rubber, polyisoprene rubber, styrene-isopropylenestyrene block copolymer (abbreviated “SIS”) and acrylate copolymer, which are used either alone or as a mixture of two or more of them. The content of the pressure-sensitive adhesive component(s) of the pressure sensitive adhesive may range from 10-50% by weight, preferably from 15-45% by weight, still more preferably 20-40% by weight.

The tackifier used for adjusting the pressure-sensitive adhesiveness includes rosin, hydrogenated rosin, and esters thereof, polyterpene resin, petroleum resin, and ester gum, etc. which are used either alone or as a mixture of two or more of them. The content of the tackifier(s) in the pressure-sensitive adhesive may be up to 40% by weight, and preferably in the range from 5 to 35% by weight, still more preferably from 15 to 30% by weight.

Further, the softener to be used in the present invention may be one or more members selected from among liquid paraffin, polybutene, liquid polyisobutylene and animal and vegetable oils. The content of the softener(s) in the pressure sensitive adhesive may range from 5 to 60% by weight, preferably from 10 to 50% by weight, still more preferably from 25 to 45% by weight.

If necessary, the pressure-sensitive adhesive may further contain one or more fillers selected from among titanium dioxide, synthetic aluminum silicate, zinc oxide, calcium carbonate, starch acrylate, silica and so forth. The content of the filler(s) in the pressure-sensitive adhesive may be up to 5% by weight, and preferably ranges from 0.1 to 4% by weight, still more preferably 1 to 3% by weight.

The sensing apparatus will contain a reporter system for measuring the presence of specific analytes. Suitable reporter systems and analytes are described herein. Generally, the reporter systems will contain multiple components for detecting and/or measuring the presence of the analyte of interest. One component of the reporter system will be attached, adhered or otherwise fixably connected to the planar backing of the sensing apparatus. Preferably, this component can be a ligand that binds the analyte of interest. Alternatively, the components of the reporter system can be contained in a porous matrix that is attached to the planar occlusive backing of the sensing apparatus. The porous matrix may be attached to the occlusive backing using the pressure-sensitive adhesive described supra, or other adhesive well know to those of skill in the art.

The porous matrix may be composed of liquid permeable material, including but not limited to cellulose derivatives such as cellulose, carboxymethylcellulose, carboxymethylcellulose salts, hydroxyethylcellulose, hydroxypropylcellulose, methylcellulose, ethylcellulose, carboxymethylethylcellulose, hydroxypropylmethylcellulose, ethylhydroxyethylcellulose, cellulose acetate, cellulose nitrate, cellulose acetate phthalate and hydroxypropymethylcellulose phthalate; porous gels such as poly-2-hydroxyethyl methacrylate, polyacrylate, polyacrylic acid and polyvinyl alcohol-polyacrylic acid composite; fibrous matrixes such as polyurethane, polyester, polyethylene, polyvinyl chloride, polyvinylidene fluoride and nylon; papers (such as nonwoven paper and filter paper); cloths (such as staple fiber, cotton, silk and synthetic fibers); and porous ceramics such as silica, alumina, titania, zirconia, and ceria, which may be used either alone, or as a mixture of two or more of them. The pore of the porous matrix will be of such a size as to allow the ingress and egress of the fluid sample while retaining the components of the reporter system. Preferably, the porous matrix may be in particulate form. Still preferably, the particles will have a size in the range of 0.1 to 250 μm and more preferably in the range of 10 to 70 μm.

The sensing apparatus may be applied to the target surface and subsequently contacted with a detection means other than those described herein to detect the analyte. The sensing apparatus may comprise a hydrogel. Suitable gelling agents for forming a hydrogel include agar, modified starches, amylopectin, carbopol, calcium, calcium lactate, cellulose gum, klucel (HPMC), natrosol, gelatin powder or sodium alginate. The gelling agents may be present in water at levels such as 1 to 4% by weight in water.

Alternatively, a hydrogel may be applied to the target surface and sufficient time allowed for analyte from the target surface to equilibrate in the gel prior to the detection step. The time may be quite short such as from 30 seconds to 5 minutes. Detection may then be carried out by applying the sensing apparatus to the gel. Alternatively, hydrogels containing analyte-specific reporter systems can be prepared by readily available techniques familiar to the ordinarily skilled artisan and used as described supra.

The invention also provides in vivo methods for detecting an analyte in an individual (as used herein, “detecting” may include qualitatively determining the presence of an analyte, as well as quantitatively measuring its concentration). The reporter system is placed in communication with sampled analyte or in contact with tissue or body fluids (e.g., interstitial fluid) of the individual suspected of containing the analyte. Such placement can be considered as permitting non-invasive or minimally invasive detection and monitoring of the analyte. The reporter system includes a fluorescence reagent for detecting the analyte. Once the reporter system is in place, it is illuminated with radiation transdermally and the fluorescence from the fluorescence reagent associated with the presence of the analyte is measured.

In the practice of the present invention, the in vivo methods generally entail two steps, a sampling (accessing) step and a detection step. The sampling, or “accessing” step can be generalized as follows. A target surface is selected and cleaned with a suitable solvent. The target surface is then disrupted in some manner sufficient to create micro-passages that allow access to a quantity of an analyte. In this regard, the analyte may be present in a fluid that flows, exudes, diffuses, perfuses, or otherwise passes from beneath the target surface, through the micro-passages to the target surface. In a preferred embodiment small sampling particles are accelerated into and/or across a target surface. These sampling particles are accelerated to a speed sufficient to penetrate the skin or mucosal layer at the target site, thereby breaching the natural barrier function of the skin or mucosal tissue and allowing access to an analyte present beneath the target surface. The target surface generally has an overall size ranging from about 0.1 to about 5 cm².

The sampling particles typically comprise an inert material. The material may be dissolvable such as commonly employed physiologically acceptable soluble materials including sugars (e.g., mannitol, sucrose, lactose, trehalose, and the like) and soluble or dissolvable polymers, e.g., swellable natural gels such as agarose. Alternatively, the sampling particles can be comprised of insoluble materials such as starch, TiO₂, calcium carbonate, phosphate salts, hydroxy-apatite, or even synthetic polymers or metals such as gold, platinum or tungsten. Insoluble materials are sloughed off with the normal skin or mucosal tissue renewal process. Preferred materials are lactose, mannitol and polyethylene glycol, such as PEG 8000.

If desired, the sampling particles can be coated with a locally active agent that facilitates the sampling step. For example, the sampling particles can be coated with or contain a pharmacological agent such as a vasoactive agent or an anti-inflammatory agent. The vasoactive agent is generally used to provide short-acting vasoactivity (e.g., up to 24 hours) in order to maximize, hasten or prolong fluid access (optimize analyte access), whereas the anti-inflammatory agent is generally used to provide local anti-inflammatory action to protect the target site. The sampling particles can also be coated with or contain an osmotically active agent to facilitate the sampling process.

The sampling particles can be delivered from a particle injection device, e.g., a needleless syringe system as described in commonly owned International Publication Nos. WO 94/24263, WO 96/04947, WO 96/12513, and WO 96/20022, all of which are incorporated herein by reference. Delivery of sampling particles from these needleless syringe systems is generally practiced with particles having an approximate size generally ranging from 0.1 to 250 μm, preferably ranging from about 10-70 μm. Particles larger than about 250 μm can also be delivered from the devices, with the upper limitation being the point at which the size of the particles would cause untoward pain and/or damage to the tissue.

The actual distance to which the delivered particles will penetrate a target surface depends upon particle size (e.g., the nominal particle diameter assuming a roughly spherical particle geometry), particle density, the initial velocity at which the particle impacts the surface, and the density and kinematic viscosity of the targeted skin tissue. In this regard, optimal particle densities for use in needleless injection generally range between about 0.1 and 25 g/cm³, preferably between about 0.9 and 1.5 g/cm³, and injection velocities generally range between about 100 and 3,000 m/sec. With appropriate gas pressure, particles having an average diameter of 10-70 μm can be readily accelerated through the nozzle at velocities approaching the supersonic speeds of a driving gas flow. Preferably, the pressure used when accelerating the particles will be less than 30 bar, preferably less than 25 bar and most preferably 20 bar or less.

Alternatively, the sampling particles can be delivered from a particle-mediated delivery device such as a so-called “gene-gun” type device that delivers particles using either a gaseous or electric discharge. An example of a gaseous discharge device is described in U.S. Pat. No. 5,204,253. An explosive-type device is described in U.S. Pat. No. 4,945,050. One example of a helium discharge-type particle acceleration apparatus is the PowderJect XR® instrument (PowderJect Vaccines, Inc., Madison, Wis.), which instrument is described in U.S. Pat. No. 5,120,657. An electric discharge apparatus suitable for use herein is described in U.S. Pat. No. 5,149,655. The disclosure of all of these patents is incorporated herein by reference.

Other methods for disrupting the target surface, in a way that micro-pathways are formed in a target skin or mucosal surface to provide access to analyte beneath the target surface, are well known in the art. The term “micro-pathways” refers to microscopic perforations and/or channels in the skin caused by pressure (water or particle injection), mechanical (micro lancets), electrical (thermal ablation, electro-poration, or electroosmosis), optical (laser ablation), and chemical methods or a combination thereof. For example, U.S. Pat. No. 5,885,211 describes five specific techniques for creating micro-pathways which entail: ablating the surface with a heat source such that tissue bound water is vaporized; puncturing the surface with a microlancet calibrated to form a micropore; ablating the surface by focusing a tightly focused beam of sonic energy; hydraulically puncturing the surface with a high pressure jet of fluid; and puncturing the surface with short pulses of electricity to form a micro-pathway. Another specific technique is described in U.S. Pat. Nos. 6,219,574 and 6,230,051, which describe a device having a plurality of microblades. The microblades are angled and have a width of 10 to 500 microns and a thickness of 7 to 100 microns and are used to provide superficial disruptions in a skin surface.

Disruption of the target surface allows access to the analyte of interest that was otherwise not accessible at the target surface. For example, disruption of the target surface can produce micro-pathways that allow a small amount of a fluid sample (e.g., a body fluid) to flow, exude or otherwise pass to the target surface via mass fluid transport, wherein the fluid contains the analyte of interest. The term “body fluid” refers to biological fluid including, but not limited to interstitial fluid, blood, lymph, sweat, or any other body fluid accessible at the surface of suitably disrupted tissue. The term “mass fluid transport” refers to the movement of fluids, such as body fluid. This term is used to distinguish over analyte transport across the disrupted surface. The mass transport aspect refers to the physical movement of the fluid (as opposed to the movement of energy, or solutes) between body fluids in tissue beneath the target surface and the surface.

Alternatively, disruption of the target surface can produce micro-pathways that simply allow access to the analyte beneath the surface from a position on the target surface itself, wherein the analyte passes to the surface essentially free of net mass fluid transport. In this regard, the analyte may simply diffuse between the tissue below the target surface and a microenvironment established at the tissue surface. The term “essentially free” refers to an insubstantial amount of mass fluid transport between the tissue and the target surface.

The term “diffusion” refers to the flux across the disrupted surface (e.g., across disrupted skin tissue) between a body fluid below the surface and the target surface itself, wherein flux occurs along a concentration gradient. Such diffusion would thus include transport of the target analyte to maintain equilibrium between the body fluid and the target surface. When the concentration of analyte is greater in the body, analyte diffusion would be toward the target surface. When the concentration of analyte is greater at the target surface, analyte diffusion would be toward the body. In addition, net diffusion of analyte from the target surface to the body fluid will occur when the concentration of analyte in the body decreases with respect to the previous measurement. Diffusion, however, is not limited to the target analyte. Certain means of measurement can generate natural byproducts of the analyte. Such byproducts can diffuse from a sensing material in contact with the target surface into the body fluid.

In methods that depend upon such “diffusional” access to the target analyte, it is preferred that an interface is applied to disrupted target surface to facilitate the establishment and maintenance of an equilibrium concentration of both analyte and any byproducts by diffusion. In this manner, the methods of the present invention permit a virtually continuous measurement during long-term monitoring without saturating the target surface with byproducts or even the analyte itself. The term “equilibrium” refers to the phenomenon in which diffusion has equalized the concentration of analyte on either side of the disrupted surface such that there is essentially no concentration gradient. Diffusion of analyte between the body fluid and the target surface allows approach to an equilibrium or steady-state condition. When concentrations of analyte change in the body, a timely dynamic change in the equilibrium enables continuous monitoring of the analyte concentration at the tissue surface. The methods of measurement or detection of the analyte contemplated herein avoid transforming or consuming a significant amount of the analyte, thereby avoiding significant reduction in the amount of analyte at the surface which could render it a sink for the analyte. However, even in a situation where a sink is created, continuous monitoring of analyte concentration can measure the rate of diffusion instead of concentration, for example in the event that the time to reach equilibrium between the target surface and the body fluid is insufficient.

After the target surface has been suitably disrupted, access to the analyte is then available at the target surface. Typically, the analyte is present in a fluid sample that has flowed, exuded or otherwise passed to the surface, substantially instantaneously, or occurring over a period of time. Alternatively, no net mass fluid transport occurs, with the analyte simply diffusing to the target surface. In methods where a particle injection device is used to disrupt the target surface, the quantity of the analyte that is made available at the target surface may be varied by altering conditions such as the size and/or density of sampling particles and the settings of the apparatus used to deliver the particles. The quantity of fluid released may often be small, such as <1 μl that is generally sufficient for detection of the analyte.

Once the analyte is accessible at the target surface, the presence and/or amount or concentration of the analyte is determined. In this regard, the target surface is contacted with a suitable sensing apparatus as described herein above. This detection step can be carried out in a continuous manner. Continual or continuous detection allows for monitoring of target analyte concentration fluctuations.

If desired, a suitable interface material may be applied to the target surface to facilitate the detection step. For example, after disrupting the surface, a gel material can be spread over the target site to provide an interface material. Examples of particularly suitable interface materials include a hydrogel, or other hydrophilic polymer, the composition of which is predominantly water for measurement of water-soluble target analytes. The hydrogel can be used with or without surfactants or wetting agents. For those methods where diffusional analyte access is used, the interface material can be formulated to provide a continuous approach to equilibrium of target analyte concentration between the interface material and the body fluid. The physical properties of the interface material are selected to maintain close association with the micro-passages or other portals. Examples of hydrogels include, but are not limited to, a 1% solution of a Carbopol® (B. F. Goodrich Co.; Cleveland, Ohio) in water, or a 4% solution of Natrosol® (Aqualon Hercules; Wilmington, Del.) in water. In some cases (e.g., diffusional analyte access) it is preferred that the interface material not withdraw a sample of body fluid, nor behave like a sink for the target analyte. In such embodiments, the composition of the interface material can be selected to render it isosmotic with the body fluid containing the target analyte, such that it does not osmotically attract body fluid. Other embodiments can comprise hydrogels including, but not limited to, poly(hydroxyethyl methacrylate) (PHEMA), poly(acrylic acid) (PAA), polyacrylamide (PAAm), poly(vinyl alcohol) (PVA), poly(methacrylic acid) (PMAA), poly(methyl methacrylate) (PMMA), poly(vinylpyrrolidone) (PVP), poly(ethylene oxide) (PEO), or poly(ethylene glycol) (PEG), avoiding polymers that can interfere with analytical methods for specific target analyte such as normal or chemically modified polysaccharides in the case of glucose measurement.

The composition of the interface material can further be selected to render it isotonic or isosmotic with the body fluid containing the target analyte, such that it does not osmotically attract mass flow of body fluid. In one embodiment, the composition can comprise a modified Ringer's-type solution to simulate interstitial fluid having a composition of NaCl (9 g/l), CaCl₂·2H₂O (0.17 g/l), KCl (0.4 g/l), NaHCO₃ (2.1 g/l), and glucose (10 mg/l). Other embodiments can comprise simpler or more complex aqueous salt compositions with osmolality ranging from 290 mOsm/kg to 310 mOsm/kg.

The interface material, e.g., the gel, may be applied to the target surface as described above and sufficient time allowed for analyte from the target surface to equilibrate in the gel prior to the detection step. The time may be quite short, such as from 30 seconds to 5 minutes. Detection may then be carried out by contacting the target surface with a reporter apparatus constructed according to the present invention.

The determination step can be generalized as follows. An initial step can entail obtaining a raw signal from a sensing device, which signal is related to a target analyte present in the biological system. The raw signal can then be used directly to obtain an answer about the analyte, for example, whether or not the analyte is present, or a direct measurement indicative of the amount or concentration of the extracted analyte. The raw signal can also be used indirectly to obtain information about the analyte. For example, the raw signal can be subjected to signal processing steps in order to correlate a measurement of the sampled analyte with the concentration of that analyte in the biological system. Such correlation methodologies are well known to those skilled in the art.

Alternatively, the sampling (“accessing”) step comprises delivery of a particulate reporter system (particles that comprise the porous matrix described supra, and which contain a reporter system for detecting the analyte of interest). As such, the reporter system will penetrate and become embedded in the target surface. Here again, the particulate reporter system particles are preferably delivered from a particle injection device, e.g., a needleless syringe system as described in commonly owned International Publication Nos. WO 94/24263, WO 96/04947, WO 96/12513, and WO 96/20022. Delivery of sampling particles from these needleless syringe systems is generally practiced with particles having an approximate size generally ranging from 0.1 to 250 μm, preferably ranging from about 10-70 μm. Particles larger than about 250 μm can also be delivered from the devices, with the upper limitation being the point at which the size of the particles would cause untoward pain and/or damage to the tissue.

The actual distance to which the delivered particles will penetrate a target surface depends upon particle size (e.g., the nominal particle diameter assuming a roughly spherical particle geometry), particle density, the initial velocity at which the particle impacts the surface, and the density and kinematic viscosity of the targeted skin tissue. With appropriate gas pressure, particles having an average diameter of 10-70 μm can be readily accelerated through the nozzle at velocities approaching the supersonic speeds of a driving gas flow. Preferably, the pressure used when accelerating the particles will be less than 30 bar, preferably less than 25 bar and most preferably 20 bar or less, and the particles will be delivered to a substantially uniform and homogenous depth, e.g., of about 1 to 50 microns below the target surface. It is a distinct advantage of this method that the particulate reporter system is thus delivered to a homogenous and substantially superficial depth in the target skin. Both the homogeneity of the particle bed, and the superficial depth of the delivered particles enable ready transdermal readings with a radiation sensing device contacted with the outer surface of the skin, and the superficial delivery further ensures that the reporter system is sloughed off with the natural turn-over of skin cells, typically within about 14-21 days. This is in distinct contrast with other systems where, for example, a reporter system is “tattooed” into the skin surface using a conventional needle to provide a substantially permanent and non-homogeneous reporter system bed. Such tattooed systems pose a safety risk as these foreign components are inserted deep into the skin tissue where they have access to vascular systems.

Alternatively, the particulate reporter system can be delivered from a particle-mediated delivery device such as a so-called “gene-gun” type device that delivers particles using either a gaseous or electric discharge. An example of a gaseous discharge device is described in U.S. Pat. No. 5,204,253. An explosive-type device is described in U.S. Pat. No. 4,945,050. One example of a helium discharge-type particle acceleration apparatus is the PowderJect XR® instrument (PowderJect Vaccines, Inc., Madison, Wis.), which instrument is described in U.S. Pat. No. 5,120,657. An electric discharge apparatus suitable for use herein is described in U.S. Pat. No. 5,149,655.

A large number of analytes may be detected according to the methods of the invention. Suitable analytes include, for example, carbohydrates (e.g., glucose, fructose, and derivatives thereof). As used herein, “carbohydrate” refers to any of the group of organic compounds composed of carbon, hydrogen, and oxygen, including sugars, starches, and celluloses. Other suitable analytes include glycoproteins (e.g., glycohemoglobin, thyroglobulin, glycosylated albumin, and glycosylated apolipoprotein), glycopeptides, and glycolipids (e.g., sphingomyelin and the ganglioside c_{M2}). Glucose is particularly preferred as an analyte due to its importance in diabetes.

Another group of suitable analytes includes ions. These ions may be inorganic or organic. Examples include calcium, sodium, chlorine, magnesium, potassium, bicarbonate, phosphate, and carbonate. The invention is also useful for detecting proteins and peptides (the latter being lower molecular weight versions of the former); a number of physiological states are known to alter the level of expression of proteins in blood and other body fluids. Included within this group are enzymes (e.g., enzymes associated with cellular death such as LDH, SGOT, SGPT, and acid and alkaline phosphatases), hormones (e.g., hormones associated with ovulation such as luteinizing hormone and follicle stimulating hormone, or hormones associated with pregnancy such as human chorionic gonadotropin), lipoproteins (e.g., high density, low density, and very low density lipoprotein), and antibodies (e.g., antibodies to diseases such as AIDS, myasthenia gravis, and lupus). Antigens and haptens are also suitable analytes.

Additionally, the invention is useful for detecting and monitoring analytes such as steroids (e.g., cholesterol, estrogen, and derivatives thereof). In the case of estrogen, the invention makes it possible to monitor menopausal patients under estrogen therapy (where estrogen levels can be quite high). The invention is also useful for detecting and monitoring substances such as theophylline (in asthma patients) and creatinine (a substance associated with renal failure).

The invention may also be used to detect and monitor pesticides and drugs. As used herein, “drug” refers to a material which, when ingested, inhaled, absorbed, or otherwise incorporated into the body produces a physiological response. Included within this group are alcohol, therapeutic drugs (e.g., chemotherapeutic agents such as cyclophosphamide, doxorubicin, vincristine, etoposide, cisplatin, and carboplatin), narcotics (e.g., cocaine and heroin), and psychoactive drugs (e.g., LSD).

The invention may also be used to detect and monitor polynucleotides (e.g., DNA and RNA). For example, overall DNA levels may be assayed as a measure of cell lysis. Alternatively, the invention could be used to assay for expression of specific sequences (e.g., HIV RNA).

As described supra, the invention features in vivo methods for detecting an analyte in an individual. According to this method, the sensing apparatus (containing a fluorescence reagent for detecting the analyte that reversibly binds to the analyte) is placed in communication with the analyte or with tissue or body fluids of the individual suspected of containing the analyte as described supra. As described supra, the preferred sensing apparatus is configured to retain the fluorescence reagent while allowing analyte to diffuse into and out of said sensor. The fluorescence reagent may include a specific binding pair, one member of which is labeled with an energy-absorbing donor molecule (which may be a fluorophore) and the other of which is labeled with an energy-absorbing acceptor molecule (which may be a fluorophore). The excited state energy level of the donor overlaps with the excited state energy level of the acceptor. The sensor is illuminated so as to i) excite the donor or ii) excite both the donor and acceptor. The fluorescence from the fluorescence reagent associated with the presence of the analyte is then measured by determining the extent to which non-radiative fluorescence resonance energy transfer (“FRET”) occurs between the donor and the acceptor upon binding. The non-radiative fluorescence resonance energy transfer, in turn, is determined by measuring i) the ratio of the fluorescence signal at two emission wavelengths, one of which is due to donor emission and the other of which is due to acceptor emission, when only the donor is excited, or ii) the ratio of the fluorescence signal due to the acceptor following donor excitation and the fluorescence signal due to the acceptor following acceptor excitation.

Basic Elements of FRET

FRET generally involves the non-radiative transfer of energy between two fluorophores, one an energy donor (D) and the other an energy acceptor (A). Any appropriately selected donor-acceptor pair can be used, provided that the emission of the donor overlaps with the excitation spectra of the acceptor and both members can absorb light energy at one wavelength and emit light energy of a different wavelength.

The method is described below with particular reference to fluorescein and rhodamine as the donor-acceptor pair. As used herein, the term fluorescein refers to a class of compounds that includes a variety of related compounds and their derivatives. Similarly, as used herein, the term rhodamine refers to a class of compounds that includes a variety of related compounds and their derivatives. Other examples of donor/acceptor pairs are NBD N-(7-nitrobenz-2-oxa-1,3-diazol-4-yl) to rhodamine, NBD or fluorescein to eosin or erythrosis, dansyl to rhodamine, acridine orange to rhodamine.

Alternatively, both the donor and acceptor can absorb light energy, but only one of them emits light energy. For example, the donor can be fluorescent and the acceptor can be nonfluorescent. It is also possible to make use of a donor-acceptor pair in which the acceptor is not normally excited at the wavelength used to excite the (fluorescent) donor; however, non-radiative FRET causes acceptor excitation.

Although the donor and the acceptor are referred to herein as a “pair,” the two “members” of the pair can, in fact, be the same substance. Generally, the two members will be different (e.g., fluorescein and rhodamine). It is possible for one molecule (e.g., fluorescein, or rhodamine) to serve as both donor and acceptor; in this case, energy transfer is determined by measuring depolarization of fluorescence.

The concept of FRET is described as follows. The absorbance and emission spectra of the energy donor, is designated A(D), and E(D), respectively, and the absorbance and emission spectra of acceptor, is designated A(A) and E(A). The absorbance and emission spectra of the donor and acceptor may differ, however, the area of overlap between the donor emission and the acceptor absorbance spectra is of importance. If, for example, excitation of the energy donor occurs at wavelength I, light will be emitted at wavelength II, the donor's emission wavelength. The acceptor, which normally emits light at wavelength III will not emit any light because the acceptor does not absorb light at wavelength I. However, if the donor emission spectra, E(D), overlaps sufficiently with the acceptor absorbance spectra, A(A), a non-radiative energy transfer process can occur resulting in an emission of light at wavelength III by the acceptor (A).

The non-radiative transfer process occurs when a donor molecule (D) absorbs the photon with a specific electric field vector, termed E. In the excited state the donor molecule will exist as a dipole with positive charge on one side and negative charge on the other. If an acceptor molecule (A) is sufficiently close to D (e.g., typically less than 100 Angstroms), an oppositely charged dipole is induced on the acceptor molecule (it is raised to an excited state). This dipole-induced dipole interaction falls off inversely as the sixth power of donor-acceptor intermolecular distance.

Classically, partial energy transfer can occur. However, this is not what occurs in FRET, which is an all or nothing quantum mechanical event. That is, a donor is not able to give part of its energy to an acceptor. All of the energy must be transferred and energy transfer can occur only if the energy levels (i.e., the spectra) overlap. When A leaves its excited state, the emitted light is rotated or depolarized with respect to the incident light. As a result, FRET manifests itself as a decrease in fluorescence intensity (i.e., decrease in donor emission) at wavelength II, an appearance of fluorescence intensity at wavelength III (i.e., an increase in sensitized emission) and a depolarization of the fluorescence relative to the incident light.

A final manifestation of FRET is in the excited state lifetime. Fluorescence can be seen as an equilibrium process, in which the length of time a molecule remains in its excited state is a result of competition between the rate at which it is being driven into this state by the incident light and the sum of the rates driving it out of this state (fluorescence and non-radiative processes). If a further non-radiative process, FRET, is added (leaving all else unchanged), decay is favored, which means donor lifetime at wavelength II is shortened.

When two fluorophores whose excitation and emission spectra overlap are in sufficiently close proximity, the excited state energy of the donor molecule is transferred by a resonance induced dipole-dipole interaction to the neighboring acceptor fluorophore. In FRET, a sample or mixture is illuminated at a wavelength which excites the donor but not the acceptor molecule directly. The sample is then monitored at two wavelengths: that of donor emissions and that of acceptor emissions. If donor and acceptor are not in sufficiently close proximity, FRET does not occur and emissions occur only at the donor wavelength. If donor and acceptor are in sufficiently close proximity, FRET occurs. The results of this interaction are a decrease in donor lifetime, a quenching of donor fluorescence, an enhancement of acceptor fluorescence intensity, and depolarization of fluorescence intensity. The efficiency of energy transfer, Eₜ, falls off rapidly as the distance between donor and acceptor molecule, R, increases. For an isolated donor/acceptor pair, the efficiency of energy transfer is expressed as:

Eₜ=1/[1+(R/R_{O})⁶] (1)

where R is the separation distance between donor and acceptor and R_{O} is the distance for half transfer. Ro is a value that depends upon the overlap integral of the donor emission spectrum and the acceptor excitation spectrum, the index of refraction, the quantum yield of the donor, and the orientation of the donor emission and the acceptor absorbance moments. Forster, T., Z Naturforsch. 4A, 321-327 (1949); Forster, T., Disc. Faraday Soc. 27, 7-17 (1959).

Because of its 1/R⁶ dependence, FRET is extremely dependent on molecular distances and has been dubbed “the spectroscopic ruler.” (Stryer, L., and Haugland, R. P., Proc. Natl. Acad. Sci. USA, 98:719 (1967). For example, the technique has been useful in determining the distances between donors and acceptors for both intrinsic and extrinsic fluorophores in a variety of polymers including proteins and nucleic acids. Cardullo et al. demonstrated that the hybridization of two oligodeoxynucleotides could be monitored using FRET (Cardullo, R., et al., Proc. Natl. Acad. Sci., 85:8790-8794 (1988)).

Using the Sensing Apparatus and FRET Reporting Systems to Measure Analyte Concentrations

The sensing apparatus and reporting systems of the present invention can be used to detect a wide range of physiological analyte concentrations. In addition, the method is reliable. Also, because the reactants are not consumed, the devices are reusable for extended periods. Moreover, the in vivo embodiments are non-invasive or minimally invasive.

In general, the sensing apparatus and the FRET reporting system is used for analyte detection is one of two ways. The first is a competitive assay in which an analogue to the analyte being detected and a ligand capable of binding to both analogue and analyte are labeled, one with a donor fluorophore and the other with an acceptor fluorophore. Thus, the analogue may be labeled with donor and the ligand with acceptor, or the analogue may be labeled with acceptor and the ligand with donor. When the labeled reagents contact analyte, analyte displaces analogue bound to ligand. Because ligand and analogue are no longer close enough to each other for FRET to occur, the fluorescence signal due to FRET decreases; the decrease correlates with the concentration of analyte (the correlation can be established in a prior calibration step).

In order to be able to reuse the fluorescence reagents, the binding between analyte and ligand should be reversible under physiological conditions. Similarly, the equilibrium binding constants associated with analyte-ligand binding and analogue-ligand binding should be such that analyte can displace analogue. In other words, analogue-ligand binding should not be so strong that analyte cannot displace analogue.

This approach is applicable to detection of carbohydrates, steroids, proteins, peptides, antigens, haptens, drugs, pesticides, theophylline, creatinine, and small organic molecules generally. In the case of carbohydrates such as glucose and fructose, suitable analogue-ligand combinations satisfying the above-described selection criteria include the following combinations: glycoconjugate-lectin, antibody-antigen, receptor-ligand, and enzyme-substrate. For example, in the case of glucose, the combination of dextran as a glucose analogue (as the glycoconjugate) and concanavalin A (as the lectin) is effective. To determine suitable combinations for other sugars, one can select a lectin that binds to the sugar and then use that lectin in combination with bovine serum albumin covalently labeled with that sugar or an analogous sugar.

In the case of analytes such as steroids, proteins, and peptides, the appropriate combination would be an analogue to the steroid, protein, or peptide, and an antibody (or antigen, where the protein or peptide is an antibody) or a receptor for the steroid, protein, or peptide. For example, in the case of steroids Haugland, R. P. (1989) Molecular Probes: Handbook of Fluorescent Probes and Research Chemicals, Molecular Probes, Eugene, Oreg., provides information on preparation of suitable analogues. Using cholesterol as a representative example, derivatives can be prepared either by covalent attachment of a fluorophore (e.g., NBD or pyrene) to the aliphatic side chain or to a hydroxyl group (e.g., using anthracene as the fluorophore). For cholesterol, the molecules thus produced are, respectively, 22-(N-(7-nitrobenz-2-oxa-1,3diazol-4-yl) amino-23,24bisnor-5-cholen-3B-ol; 1-pyrenemethyl 3B-hydroxy-22,23-bisnor-5-cholenate; and cholesteryl anthracene-9-carboxylate. The steroid can also be conjugated to a carrier protein or other macromolecule that would also be fluorescently tagged with donor or acceptor. The conjugation would again proceed via either the aliphatic side chain or the hydroxyl group.

Similar considerations apply in the case of glycoproteins, glycopeptides, and glycolipids. In the case of glycosylated hemoglobin, FRET between a labeled lectin and the heme itself could be measured (this would manifest itself as a quenching of fluorescence).

The second approach using the sensing apparatus and FRET reporting system is to select two ligands that bind to different portions (sites) of an analyte molecule; in addition to being spatially different, the portions may be chemically different as well. This approach is applicable to detection of antigens, haptens, steroids, proteins, peptides, drugs, pesticides, theophylline, creatinine, and large organic molecules generally. The ligands could be two antibodies, two cell receptors, or an antibody and a cell receptor. For example, in the case of hormones such as HCG, FSH, and LSH the labeled ligands could be antibodies or cell receptors that bind to different portions of the hormone molecule.

One variation of this second approach is to detect antibodies such as anti-DNA antibodies in lupus patients by encapsulating two fluorescent DNA fragments, one labeled with donor and the other with acceptor, and then measuring FRET (which would occur if the antibody of interest were present and crosslinked the labeled fragments).

Another variation involves labeled oligonucleotide probes. As described in Cardullo, R., et al., Proc. Natl. Acad. Sci., 85:8790-8794 (1988), the hybridization of two oligodeoxynucleotides can be monitored using FRET in conjunction with such probes. In this way, specific DNA sequences can be determined.

To assay overall DNA levels, reagents that bind non-specifically to DNA or RNA are used. Examples of such reagents include fluorescent intercalating dyes that show dramatic spectral shifts upon binding.

In yet another variation, a single material is labeled with both donor and acceptor fluorophores. The fluorescence change associated with the conformational change in the material upon binding to analyte is used as an indication of analyte presence. For example, the analyte may be a helical DNA molecule and the fluorescence reagent is a material labeled with donor and acceptor fluorophores that binds to the DNA. Binding changes the separation distance between the donor and acceptor, and thus the signal detected by FRET.

Using the Sensing Apparatus and FRET to Measure Glucose Concentrations

One aspect of the present invention relates to a sensing apparatus and a FRET reporting system in a method of detecting and quantifying glucose in a body fluid. The present method relies on the process of non-radiative fluorescence resonance energy transfer (FRET) to determine the occurrence and extent of binding between members of a specific binding pair that is competitively decreased by glucose. Members of the binding pair are a ligand (e.g., a lectin, monoclonal antibody) and a carbohydrate-containing receptor (referred to as a glycoconjugate), which binds specifically to the ligand in competition with glucose. Both the ligand and the glycoconjugate are fluorescently labeled, but typically are not labeled with the same fluorophore. They are brought into contact with a sample as described supra (e.g., interstitial fluid) in which glucose concentration is to be determined.

The present sensing apparatus and FRET reporting system and method are particularly useful in the day-to-day monitoring of glucose concentrations in individuals in whom glucose homeostasis is compromised (e.g., diabetic or hypoglycemic individuals) and in biomedical research.

Using the sensing apparatus and FRET to measure glucose concentrations in body fluid is described as follows. One macromolecule of the reporting system (designated M) includes a covalently bound fluorophore and is referred to as a glycoconjugate (e.g., dextran). A second macromolecule of the reporting system (designated L) includes a ligand that has a high degree of specificity for glucose (e.g., concanavalin A) and a fluorophore that is generally not the same fluorophore as that on the first macromolecule.

One of these fluorophores is chosen to be a donor and the other is an acceptor as described previously. For the purposes of this illustration, the donor molecule has been placed on the glycoconjugate and the acceptor has been placed on the ligand. The association can then be diagrammed as:

DM+AL→DM−LA,

where DM stands for Donor-Macromolecule, AL stands for Acceptor-Ligand, and DM-LA represents the association between the glycoconjugate present in the first complex and the ligand present in the second complex. Upon association, the two macromolecules are now close enough to allow energy transfer between the donor and the acceptor to occur.

Spectra are collected by exciting fluorescein at 472 ηm and scanning the emission from 500-650 ηm. Typically, fluorescence intensities are monitored at the emission maxima for fluorescein (about 520 ηm) and rhodamine (about 596 ηm). The measure of energy transfer is the ratio of fluorescence intensities at 520 ηm and 596 m (i.e., FI 520/FI 596) as a function of glucose concentration or the quenching of fluorescein at 520 ηm as measured by a fluorimeter.

The presence of free glucose introduces a competitive inhibitor into the formula because free glucose competes with the conjugated dextran for the ligand. Thus, increasing concentrations of glucose produces a decrease in the amount of ligand binding to the glycoconjugate. At relatively low concentrations of glucose, the transfer efficiency will remain high, since little of the macromolecular association will be affected. At high concentrations of glucose, the transfer efficiency will be low, due to the fact that the glucose has successfully competed the ligand off of the dextran.

The methods of the subject invention can be used to detect and quantify glucose in samples of a size appropriate for obtaining from an individual (e.g., 0.1-10 μl).

Based on the methods of the subject invention, a number of reporter systems can be constructed to detect glucose concentration in blood in vivo. These reporter systems can remain active for extended periods of time (e.g., one day or more) before having to be replaced. Typically, a new sensing apparatus containing a reporter system is applied to the skin or mucosal surface on a daily basis.

Accordingly, a novel sensing apparatus and monitoring methods are disclosed. Although preferred embodiments of the subject invention have been described in some detail, it is understood that obvious variations can be made without departing from the spirit and the scope of the invention as defined by the appended claims.

What is claimed is:

1. A sensing apparatus, comprising:
(a) a substantially planar occlusive backing; and
(b) a reporter system that absorbs or emits a detectable radiation, said reporter system attached, adhered, or otherwise connected to a first planar surface of the occlusive backing, wherein said reporter system binds an analyte of interest and the ability of said reporter system to absorb or emit radiation is detectably altered in a concentration dependent manner when the analyte is bound to said reporter system.

2. The apparatus of claim 1 wherein said occlusive backing has sufficient drape characteristics to allow positioning of said apparatus over a skin or mucosal surface.

3. The apparatus of claim 1 wherein the reporter system comprises a specific binding pair having a first component that is an analyte-specific binding ligand comprising a first light-absorbing material, and a second component that binds to the binding ligand of said first component and comprises a second light-absorbing material, wherein:
(a) binding of said second component to the first component is reversible;
(b) the analyte binds to the first component in a competitive manner, thereby displacing said second component; and
(c) displacement of the second component produces a detectable alteration in the energy transfer between the first component and the second component, wherein said alteration is proportional to the concentration or amount of said analyte that binds to the first component.

4. The apparatus of claim 3 wherein the binding ligand is a glucose binding ligand and the analyte of interest is glucose.

5. The apparatus of claim 4 wherein said ligand is concanavalin-A.

6. The apparatus of claim 4 wherein the second component comprises a dextran glycoconjugate.

7. The apparatus of claim 3 wherein the first and second light-absorbing materials are fluorophores.

8. The apparatus of claim 3 wherein the detectable alteration in the energy transfer between the first component and the second component comprises a non-radiative fluorescence resonance energy transfer between said first and second light-absorbing materials.

9. The apparatus of claim 3 wherein the first component of the specific binding pair is tetramethylrhodamine isothiocyanate-concanavalin A (“TRITC-ConA”) and the second component of the specific binding pair is fluorescein isothiocyanatedextran (“FITC-dextran”).

10. The apparatus of claim 1 wherein the reporter system is disposed within a polymer matrix having a pore size that allows for ingress and egress of a fluid containing or suspected of containing said analyte of interest.

11. The apparatus of claim 10 wherein said polymer matrix is in particulate form.

12. The apparatus of claim 11 wherein the polymer matrix is in the form of porous particles having a size predominantly in the range of 0.1 to 250 μm.

13. A method for detecting the presence or amount of an analyte present beneath a target skin or mucosal surface of an individual, said method comprising:
(a) disrupting the target surface to create one or more passages in that surface sufficient to allow said analyte to flow, exude, diffuse or otherwise pass from beneath the target surface to the target surface;
(b) placing the sensing apparatus of claim 1 in contact with the target surface and allowing the reporter system to contact analyte that has passed to the target surface; and
(c) detecting an alteration in the ability of the reporter system to absorb or emit radiation, thereby obtaining a signal indicative of the presence and/or amount of analyte present beneath the target surface.

14. The method of claim 13 wherein the target surface is disrupted by accelerating particles into said target surface.

15. The method of claim 14 wherein the particles have a size ranging from 0.1-250 μm.

16. The method of claim 15 wherein the particles have a size ranging from 10-70 μm.

17. The method of claim 13 wherein the analyte is glucose.

18. A method for quantifying glucose present in a body fluid beneath a target surface, said method comprising:
(a) accelerating particles into the target surface, wherein acceleration of said particles into the target surface is effective to allow passage of glucose from beneath the target surface to the target surface;
(b) contacting the glucose present at the target surface with a specific binding pair comprising a first component which is a glucose binding ligand containing a first light-absorbing material, and a second component which is a glycoconjugate containing a second light-absorbing material, the excited state energy level of the first light-absorbing material overlapping with the excited state energy level of the second light-absorbing material, said ligand and said glycoconjugate being chosen such that they reversibly bind to each other thereby allowing glucose present at the target surface to displace said glycoconjugate and competitively bind to said ligand;
(c) determining the extent to which non-radiative fluorescence resonance energy transfer occurs between the first light-absorbing and the second light-absorbing material in the presence of the glycoconjugate displaced by glucose and the ligand reversibly bound to glucose; and
(d) comparing the result of step (c) with the relationship between the extent of non-radiative energy transfer between the first light-absorbing material and the second light-absorbing material and glucose concentration in the body fluid determined in a calibration step.

19. The method of claim 18, wherein acceleration of said particles into the target surface serves to increase the permeability of the target surface.

20. The method of claim 18, wherein the particles are accelerated toward the target surface using a needleless syringe device.

21. The method of claim 18, wherein the particles are accelerated toward the target surface at a velocity of about 100 to 2,500 m/sec.

22. The method of claim 18, wherein the particles have a size predominantly in the range of 0.1 to 250 μm.

23. The method of claim 18, wherein the particles penetrate the skin to a depth in the range of 1 to 50 μm.

24. A method for detecting the presence or amount of an analyte present beneath a target skin surface of an individual, said method comprising:
(a) providing a particulate reporter system, wherein said reporter system binds the analyte of interest and the ability of said reporter system to absorb or emit radiation is altered in a concentration-dependent manner when said analyte is bound to said reporter system, and said particulate reporter system is comprised of particles having a size ranging from 0.1-250 μm;
(b) administering said reporter system into the target skin surface such that said particulate reporter system is delivered to a substantially uniform and homogenous depth within said skin;
(c) allowing the reporter system to contact the analyte; and
(d) detecting an alteration in the ability of said reporter system to absorb or emit radiation thereby obtaining a signal indicative of the presence or amount of analyte present beneath said target skin surface.

25. The method of claim 24 wherein said particulate reporter system is delivered using a needleless syringe.

26. The method of claim 25 wherein said particulate reporter system is accelerated toward the target skin surface at a velocity of about 100 to 2,500 m/s.

27. The method of claim 25 wherein said particulate reporter system is delivered at a depth of about 1-50 μm beneath said target skin surface.

28. The method of claim 24 wherein the particles have a size ranging from 10-70 μm.

29. The method of claim 24 wherein said reporter system comprises a specific binding pair having a first component that is an analyte-specific binding ligand comprising a first light-absorbing material, and a second component that binds to the binding ligand of said first component and comprises a second light-absorbing material, wherein:
(a) binding of said second component to the first component is reversible;
(b) the analyte binds to the first component in a competitive manner, thereby displacing said second component; and
(c) displacement of the second component produces a detectable alteration in the displacement of the second component and produces a detectable alteration in the energy transfer between the first component and the second component, wherein said alteration is proportional to the concentration or amount of said analyte that binds to the first component.

30. The method of claim 29 wherein the binding ligand is a glucose binding ligand and the analyte of interest is glucose.

31. The method of claim 29 wherein said ligand is concanavalin-A.

32. The method of claim 29 wherein the second component comprises a dextran glycoconjugate.

33. The method of claim 29 wherein the first and second light-absorbing materials are fluorophores.

34. The method of claim 29 wherein the detectable alteration in the displacement of the second component produces a detectable alteration in the energy transfer between the first component and the second component comprising a non-radiative fluorescence resonance energy transfer between said first and second light-absorbing materials.
